(19) United States
(12) Patent Application Publication
(10) Pub. No.: US 2026/0248872 A1
(43) Pub. Date: Aug. 27, 2026

(54) WITHANIA SOMNIFERA COMPOSITION FOR IMPROVING MEMORY AND COGNITION

(71) Applicant: ARJUNA NATURAL PRIVATE LIMITED, Aluva (IN)

(72) Inventor: Benny ANTONY, Aluva (IN)

(73) Assignee: ARJUNA NATURAL PRIVATE LIMITED, Aluva (IN)

(21) Appl. No.: 19/161,366

(22) PCT Filed: Mar. 9, 2024
(86) PCT No.: PCT/IB2024/052289

§ 371 Date: Sep. 2, 2025

(30) Foreign Application Priority Data
Mar. 10, 2023 (IN) 202341016031

(51) Int. Cl. A61K 36/81; A61K 31/585; A61K 31/704; A61P 25/28

(52) U.S. Cl. CPC A61K 36/81; A61K 31/585; A61K 31/704; A61P 25/28; A61K 2236/333; A61K 2236/35; A61K 2236/39; A61K 2236/55

(57) ABSTRACT

The present invention relates to a medicinal composition derived from Withania somnifera extract comprising withanolides and sominone wherein said sominone in the extract is standardized in the range of 0.1-30%. Withanolides in the said composition are in the range of 10-60%. It is further comprising withanolide glycosides and withaferin A. Medicinal composition along with pharmaceutically acceptable excipients are formulated into a dosage form. It also discloses a method of preparation of said medicinal composition. Said composition is used to improve memory and cognition when administered to subjects in the appropriate dosage. Said composition is capable of enhancing the bioavailability of its constituents.

### FIELD OF INVENTION

The present invention relates to a medicinal composition derived from Withania somnifera extract comprising withanolides and sominone. The composition further comprises, withanolide glycosides and withaferin A. Said composition is used to improve memory and cognition when administered to subjects in the appropriate dosage.

### BACKGROUND

Memory is a fundamental mental process, and without memory we are capable of nothing but simple reflexes and stereo type behaviour. Stress, anxiety or depression can cause forgetfulness, confusion, difficulty in concentrating and other problems that disrupt daily activities. Long-term potentiation (LTP), a ground-breaking discovery made in 1966, suggested that memories might be encoded in the potency of synaptic signals between neurons. As a result, we began to comprehend memory as a neurochemical phenomenon. Recent advances in science and technology indicate that the notion of memory has to be expanded and unified. Thus, many people now believe that cognition is also extended, meaning that memory may be stored somewhere other than in the brain.

Modern psychology defines memory as the ability to encode, store, and retrieve information. Three key kinds of memory have been identified by psychologists: sensory, short-term, and long-term. Each of these types of memory has unique properties. The term “sensory memory” describes the ability to remember details from the senses. Information that is processed quickly is referred to as short-term memory. This processing is carried out in working memory (WM). The central executive (attention control), the visuospatial sketchpad (creates and maintains a visuospatial representation), the phonological buffer (stores and consolidates new words), and the episodic buffer are the four components of working memory that process information stores and integrates information from different sources. We can keep information in our long-term memory for a very long time. A conscious (explicit memory) or unconscious retrieval of this knowledge is possible (implicit memory). Both episodic memory (time-related experiences) and semantic memory (concepts and meanings) are components of explicit memory. Associative memory (classical and operant conditioning), non-associative memory (sensitization and habituation), procedural memory (motor and executive abilities), and priming (a primary stimulus influencing a secondary one) are all components of implicit memory.

With advancing age, cognition and memory typically deteriorate. Although wide variation in cognitive aging can be seen even in healthy individuals, it may be linked to chronic somatic and neurodegenerative disorders. Mild cognitive impairment (MCI) is a neurocognitive disorder which involves cognitive impairments beyond those expected based on an individual's age and education but which are not significant enough to interfere with instrumental activities of daily living. According to a recent 10-year study, cognitive impairment frequently starts between the ages of 45 and 55. Mild cognitive impairment is classified into two subtypes: amnestic and non-amnestic. Amnestic mild cognitive impairment is clinically significant memory impairment that does not meet the criteria for dementia. Non-amnestic mild cognitive impairment is characterized by a subtle decline in functions not related to memory, affecting attention, use of language, or visuospatial skills.

Mild cognitive impairment (MCI) may be caused due to alteration in the brain triggered during early stages of Alzheimer's disease or other forms of dementia.

For thousands of years, traditional alternative medicine systems like Ayurveda and Traditional Chinese Medicine (TCM) have prescribed herbal treatments to improve memory and cognition.

Ashwagandha is also known as Withania somnifera (WS) (L.) Dunal, is a popular medicine that is categorised as “adaptogen” or a Rasayana in the Indian Ayurvedic pharmaceutical system.

In Ayurveda, the roots of W. somnifera are used to prepare many herbal medicines. The biologically active chemical constituents of Withania somnifera are alkaloids (isopelletierine, anaferine), steroidal lactones (withanolides, withaferins), saponins containing an additional acyl group (sitoindoside VII and VIII), and withanolides with glucose at carbon 27 (sitoindoside XI and X). There have also been reports on other constituents from plants of the Withania genus, namely, fatty acids and volatile compounds.

Several constituents of Withania somnifera and its activities are known. But a medicinal composition derived from Withania somnifera extract comprising withanolides and sominone is not disclosed in any of the prior art. Further said composition also contains withanolide glycosides and withaferin A. Said composition is found to improve memory and cognition when administered to subjects in the appropriate dosage.

### SUMMARY

Memory is a cornerstone of cognitive function, without which our capabilities reduce to mere reflexes and stereotypical behaviour. Conditions such as stress, anxiety, or depression can lead to forgetfulness, confusion, and difficulty concentrating, disrupting our daily activities. We have formulated a medicinal composition derived from Withania somnifera extract, comprising withanolides and sominone, aimed at enhancing memory function.

Said composition is used to improve memory and cognition when administered to subjects in the appropriate dosage. The composition further comprises withanolide glycosides and withaferin A.

Accordingly, we disclose a medicinal composition derived from Withania somnifera extract comprising withanolides and sominone wherein said sominone in the extract is standardized in the range of 0.1-30%. In the said medicinal composition withanolides are in the range of 10-60%. Said composition further comprises withanolide glycosides and withaferin A, preferably in the range of 10-25%, and 2-10% respectively. According to one embodiment of the invention, said medicinal composition comprises not less than 2% sominone, not less than 20% withanolide glycosides, not less than 6% withaferin A and not less than 30% withanolides. In one more embodiment, said medicinal composition comprises 2-3% sominone, 20-25% withanolide glycosides, 6-8% withaferin A and 30-40% withanolides. In yet another embodiment under the invention, said medicinal composition comprises 2.3% sominone, 23.5% withanolide glycosides and 6.5% withaferin A, and 34.9% withanolides.

The medicinal composition when administered to subjects is found to enhances the bioavailability of its constituents.

Invention further relates to a dosage form comprising a medicinal composition derived from Withania somnifera extract having withanolides and sominone wherein said sominone in the extract is standardized in the range of 0.1-30%. Said dosage form is having withanolides in the range of 10-60%. It is further comprising of withanolide glycosides and withaferin A, preferably in the range of 10-25%, and 2-10% respectively. Said medicinal composition comprises 2-3% sominone, 20-25% withanolide glycosides, 6-8% withaferin A and 30-40% withanolides. Said medicinal composition comprises not less than 2% sominone, not less than 20% withanolide glycosides, not less than 6% withaferin A and not less than 30% withanolides. In one embodiment under the invention, said dosage form comprises 2.3% sominone, 23.5% withanolide glycosides and 6.5% withaferin A, and 34.9% withanolides. The dosage form comprising said medicinal composition is used to enhances the bioavailability of its constituents.

Invention also relates to a method of preparation of medicinal composition derived from Withania somnifera comprising:
a) cleaning the Ashwagandha plant parts (roots and leaves);
(b) drying the cleaned plant parts and powdering it;
(c) extracting the powdered parts using a polar solvent;
(d) collecting the supernatant after extracting and followed by concentrating;
(e) subjecting said concentrate to liquid-liquid extraction using nonpolar solvent;
(f) separating the aqueous and solvent part after the liquid-liquid extraction;
(g) subjecting the aqueous part in step (f) to column purification using water and methanol;
(h) collecting the water and methanol elute and keeping aside;
(i) subjecting the solvent part collected in step (f) to column purification by eluting with hexane-methanol in different ratios and collecting different fractions; and
(j) blending methanol elute of step (h) and one of the fractions containing higher Ashwagandha composition resulting said composition.

The blending ratio of methanol elute of step (h) and one of the fractions containing higher Ashwagandha composition of step (i) ranges from 20:1 to 2:1 and preferably 9:1.

Said dosage form comprising the above mentioned medicinal composition is used for:
1. improving learning and behavioral activity;
2. improving immediate memory, general memory, working memory and visuospatial memory;
3. improving verbal episodic memory;
4. improving verbal learning and memory abilities;
5. improving visual memory and recognition abilities;
6. enhancing working memory and cognitive processing abilities;
7. enhancing the visuospatial working memory and attentional abilities by improvement in Spatial span score;
8. enhancing the visual memory and visuospatial abilities;
9. improving individual's spatial visualization abilities assessed by increase in Shepard Mental Rotation Task score; and
10. improving cognitive impairment by improving in Montreal cognitive assessment (MoCA) score and Mini-Mental State Examination (MMSE) scores.

The medicinal composition of the invention is made into dosage form selected form of capsule, tablet, mini tablet, granule, sachet, powder, paste, infusion, injection, ampoule, solution, suspension, emulsion, pills, or cream. The dosage for administering to a mammal especially to human subject is ranging from about 30 mg to about 2000 mg per day. The dosage form further comprises pharmaceutically acceptable excipients selected from the group comprising diluents, binders, wetting agents, disintegrants, and lubricants and mixture thereof.

### BRIEF DESCRIPTION OF DRAWINGS

These and other features aspects, and advantages of the present invention will be better understood when the detailed description is read along with the following accompanying drawing.

FIG. 1 represents Montreal Cognitive Assessment (MoCA) score after administering Ashwagandha extract and Placebo.

FIG. 2 represents Mini-Mental State Examination (MMSE) score after administering Ashwagandha extract and Placebo.

FIG. 3 represents Shepard Mental Rotation Task score after administering Ashwagandha extract and Placebo.

FIG. 4A represents Wechsler Memory Scale-III (WMS-III)—Immediate Memory after administering Placebo.

FIG. 4B represents Wechsler Memory Scale-III (WMS-III)—Immediate Memory after administering Ashwagandha extract.

FIG. 5A represents Wechsler Memory Scale-III (WMS-III)—General Memory after administering Placebo.

FIG. 5B represents Wechsler Memory Scale-III (WMS-III)—General Memory after administering Ashwagandha extract.

FIG. 6A represents Wechsler Memory Scale-III (WMS-III)—Working Memory after administering Placebo.

FIG. 6B represents Wechsler Memory Scale-III (WMS-III)—Working Memory after administering Ashwagandha extract.

FIG. 7A represents Wechsler Memory Scale-III (WMS-III)—Visuospatial processing and response after administering Placebo.

FIG. 7B represents Wechsler Memory Scale-III (WMS-III)—Visuospatial processing and response after administering Ashwagandha extract.

### DETAILED DESCRIPTION

The invention is directed towards a medicinal composition obtained from Withania somnifera (Ashwagandha) and its use for improving memory and cognition. The Withania somnifera extract composition comprises withanolides and sominone. Withanolides in Withania somnifera extract composition is the sum of withanolide glycosides and withanolide aglycones. The Withania somnifera extract composition comprises 10-60% withanolides and sominone in the extract is standardized in the range of 0.1-30%. The Withania somnifera extract composition comprises withanolides and sominone and said sominone in the extract is standardized with at least 2%. The composition further comprises withanolide glycosides and withaferin A. The invention provides a process for preparing the Ashwagandha extract.

The word “Withania somnifera” and “Ashwagandha” mean the same thing and may be used interchangeably throughout the specification. The term “Withania” referred to plants from the group but not limited to Acnistus, Datura, Deprea, Dunalis, Iochroma, Jaborosa, Lycium, Nicandra, Physalis, Salpichroa, Tubocapsicum, Discopodium, Trechonaetes, Withania, Witheringia, and any plant from the genus withania or a plant that contains Withanolides.

As used herein, the term “subject” refers to the target of administration, it can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and new-born subjects, whether male or female, are intended to be covered unless otherwise specified.

Ashwagandha plant material contain negligible level of sominone. Sominone, an aglycone of withanoside IV., was identified as an active metabolite after oral administration of withanoside IV. Sominone can also be synthetically prepared from dehydroepiandrosterone on the basis of an RCM strategy. Sominone is an important metabolite of withanosides, but there is no report on standardization of a memory-enhancing formulations of Ashwagandha with Sominone as a marker. According to the present invention, a medicinal composition derived from Ashwagandha extract is comprising of withanolides and sominone. The composition further comprises withanolide glycosides, and withaferin A. The composition when administered to subjects is found to improve the memory and cognitive ability.

The medicinal composition derived from Withania somnifera extract is comprising of 1% to 20% sominone, 10% to 25% withanolide glycoside, 2% to 10% withaferin A, and 10-40% withanolides.

Said medicinal composition derived from Withania somnifera extract preferably comprising of 10-60% withanolides, 0.1% to 30% sominone, 10% to 25% withanolide glycoside and 2% to 10% withaferin A.

In yet another embodiment, said medicinal composition comprises not less than 2% sominone, not less than 20% withanolide glycosides, not less than 6% withaferin A and not less than 30% withanolides. In another embodiment under the invention, the medicinal composition is derived from Withania somnifera extract comprises 2-3% sominone, 20-25% withanolide glycosides, 6-8% withaferin A and 30-40% withanolides.

In another embodiment under the invention, the medicinal composition derived from Withania somnifera extract comprises 2.3% sominone, 13.5% withanolide glycosides and 5.4% withaferin A, withanolides 20.5%.

In one more embodiment, the medicinal composition derived from Withania somnifera extract comprises 2.3% sominone, 23.5% withanolide glycosides, 6.5% withaferin A, and 34.9% withanolides

The present invention also provides a process of extraction of Ashwagandha comprising withanolides and sominone wherein sominone in the extract is standardized to contain at least 2% sominone.

The present invention also provides a process for extraction of Ashwagandha composition with withanolides and at least 2% sominone comprises the following steps:
(a) Cleaning the Ashwagandha plant parts (roots and leaves).
(b) Drying the cleaned plant parts and powdering it.
(c) Extracting the powdered parts using a polar solvent.
(d) Collecting the supernatant after extracting and followed by concentrating.
(e) Subjecting said concentrate to liquid-liquid extraction using nonpolar solvent.
(f) Separating the aqueous and solvent part after the liquid-liquid extraction.
(g) Subjecting the aqueous part in step (f) to column purification using water and methanol.
(h) Collecting the water and methanol elute and keeping aside.
(i) Subjecting the solvent part collected in step (f) to column purification by eluting with hexane-methanol in different ratios and collecting different fractions.
(j) Blending methanol elute of step (h) and one of the fractions containing higher percentage of actives obtained in step (i) to form Ashwagandha composition.
   The blending ratio of methanol elute and fraction containing higher percentage of actives ranges from 20:1 to 2:1. Preferred blending ratio is 9:1.

The adsorbing column can be silica, SP700, HP20, HP2MGL, SA10A, WA10, CRB03, CRB05, CR20, XAD 7HP, FP66, SK1B, or SP825L.

The polar solvents used for extracting ashwagandha are selected from water, lower alcohols such as methanol, ethanol, acetone, isopropyl alcohol, etc., ketones such as acetone, methyl ethyl ketone, diethyl ketone, ethyl butyl ketone, etc., ethers such as dimethyl ether, diethyl ether, tetrahydrofuran (THF) and combinations thereof.

The non-polar solvents used for extracting ashwagandha are selected from a group of chloroform, hexane, toluene, ethyl acetate, carbon tetrachloride, and combinations thereof.

The polar solvent used in the above-mentioned process is preferably methanol. The nonpolar solvent used in the abovementioned process is preferably Ethyl acetate.

In another embodiment, the extract of Withania somnifera along with pharmaceutically acceptable excipients are formulated into an oral dosage form.

Accordingly, the pharmaceutically acceptable excipients are selected from the group comprising diluents, binders, wetting agents, disintegrants, and lubricants and mixture thereof as the delivery systems.

The medicinal composition derived from Withania somnifera extract is in the dosage form selected from capsule, tablet, mini tablet, granule, sachet, powder, paste, infusion, injection, ampoule, solution, suspension, emulsion, pills, or cream.

Further, a dosage form of the medicinal composition derived from Withania somnifera extract is disclosed for administering in a dosage ranging from about 30 mg to about 2000 mg per day to a subject.

In one embodiment, said dosage of the medicinal composition derived from Withania somnifera extract is comprising of 10-60% withanolides and 0.1-30% sominone. According to another embodiment, said dosage of the medicinal composition derived from Withania somnifera extract is comprising of 10-60% withanolides and at least 2% sominone.

The medicinal composition when administered to subjects is capable of enhancing the bioavailability of its constituents. Said medicinal composition for enhancing the bioavailability of constitutes is comprising of Ashwagandha extract with 10-60% withanolides and 0.1-30% sominone. A method of enhancing the bioavailability of constitutes by administering Ashwagandha extract with 10-60% withanolides and 0.1-30% sominone is also disclosed.

Efficacy of the Ashwagandha composition on learning and memory (behavioural) activity is studied in rats. The behavioural test is conducted at the end of the treatment by using the Elevated plus maze apparatus, Y maze model, Cooks pole climbing method, and Morris water maze method. The rats treated with Ashwagandha extract have shown transfer latency almost similar to the standard group Piracetam.

In a pole climbing test, early escape to shock free zone (avoidance of shock) indicates retention of learned task. The rats treated with Ashwagandha extract have showed an impressive memory retention effect and it is almost similar to the standard drug Piracetam.

The retention of learned task as well as number of arm entries are observed using Y-Maze in rats. The retention latency is significant in the Ashwagandha extract-treated group. The data on the number of arm entries has also shown a significant difference for Ashwagandha extract treated rats.

Effect on scopolamine-induced amnesia in Morris Water Maze (MWM) experiment showed that pre-treatment with Ashwagandha extract in rats exhibited amelioration of scopolamine-induced amnesia. The ameliorating effect of Ashwagandha extract is evident from the significant decrease in the escape latency as compared to scopolamine-treated group on day 10. Piracetam also significantly reversed the scopolamine induced amnesia as evident by the decreased escape latency as compared to the scopolamine-treated group.

The transfer latency of rats of the scopolamine-treated group got significantly enhanced after injection of scopolamine. All the extracts as well as standard drug Piracetam are able to reverse the amnesic effect of scopolamine significantly. The rats treated with Ashwagandha extract have shown the transfer latency almost similar to the standard group Piracetam.

In another embodiment, the effect of Ashwagandha composition in improving memory in adults with mild cognitive impairment is studied. The primary outcome measures are Wechsler Memory Scale (WMS), Visuospatial processing and response, and Shepard Mental Rotation Task. Secondary outcome measures are Wisconsin Card Sorting Test (WCST), Eriksen flanker task, Trail-Making Test (Part A), Mackworth Clock Test, Montreal cognitive assessment (MOCA) and Mini mental state Examination (MMSE).

### Wechsler Memory Scale (WMS)

The WMS-III is an individually administered battery of 11 subtests of learning, memory and working memory. Six of the subtests are included in the core battery, and five subtests are considered optional I supplemental. Only six of the WMS-III subtests are considered primary. (Logical Memory I and II, Verbal Paired Associates I and II, Letter-Number Sequencing are in Auditory presentation and Faces I and II, Family Pictures I and II, and Spatial Span are in visual presentation.), and all must be administered in order to calculate the WMS-III index scores. The primary subtests can be administered in 30-35 minutes. The Information and Orientation, Word Lists I and II, and Mental Control, Digit Span are optional in auditory presentations and Visual Reproduction I and II subtests are considered optional in visual presentation and none of these optional indexes contribute to the WMS-III index scores but are used to obtain supplementary information. The Family Pictures subtest is new to the WMS-III, is purported to be the visual analogue to the Logical Memory subtest. The WMS-III Faces subtest uses a recognition paradigm to assess visual immediate and visual delayed memory. The Letter-Number Sequencing subtest is new to the WMS-III and is a measure of auditory working memory. The WMS-III provides for the calculation of eight primary index scores and four Auditory Process Composite scores.

### Visuospatial Processing and Response

Visual-spatial processing is the ability to tell where objects are in space. That includes your own body parts. It also involves being able to tell how far objects are from you and from each other.

These skills usually are assessed by paper-and-pencil copying of two- or three-dimensional figures.

### Shepard Mental Rotation Task

The experiment specifically tested mental rotation on three-dimensional objects. Each subject was presented with multiple pairs of three-dimensional, asymmetrical lined or cubed objects. The experiment was designed to measure how long it would take each subject to determine whether the pair of objects was indeed the same object or two different objects. Increase in Shepard Mental Rotation Task score shows individual's spatial visualization abilities.

### Wisconsin Card Sorting Test (WCST)

The Wisconsin Card Sorting Test (WCST) is a neuropsychological test that is frequently used to measure such higher-level cognitive processes as attention, perseverance, WM, abstract thinking, CF, and set shifting. It is particularly used in clinical fields to measure perseverative behaviors that refer to an individual's insistence on wrong behavior. Moreover, to be able to change category, one needs to have high intellectual flexibility and ability in concept formation.

The WCST consists of two card packs having four stimulus cards and 64 response cards in each. Each card measures 7×7 cm, and there are various geometric shapes in different columns and numbers. The participants are expected to accurately sort every response card with one of four stimulus cards through the feedback (right or wrong) given to them based on a rule. Among various versions, the version of WCST with 128 cards developed by Heaton was used in this study. The test was applied individually, and 12 scores were obtained (Heaton, Chelune, Talley, Kay, & Curtiss, 1993). A reliability study could not be conducted due to the nature of the test. The validity studies, on the other hand, were conducted on both sick and healthy groups, and it was shown that the test was valid for a Turkish sample (Kafadar, 2004).

### Eriksen Flanker Task

Executive functions are a key dimension of cognition and are comprised by components such as selective attention, response inhibition, and working memory. Different tests have been well established to examine individual executive Functions. The flanker task requires (spatial) selective attention and executive control. In this task, irrelevant stimuli have to be inhibited in order to respond to a relevant target stimulus. Eriksen flanker task is a set of tests to assess selective attention and inhibitory function. In this task, the target is positioned in the center and is flanked by non-target stimuli. The individual is requested to press the left or right arrow key according to the target's direction.

### Trail-Making Test (Part A)

The Trail Making Test is a neuropsychological test of visual attention and task switching Assessment of subject's visual attention and task switching abilities can provide information about visual search speed, speed of processing, and mental flexibility as well as executive functioning. Part A is an 8″×11″ page on which the numbers 1-25 are scattered within circles. The patient is instructed to draw lines connecting the numbers in order as quickly as possible. Results for TMT A is reported as the number of seconds required to complete the task; therefore, higher scores reveal greater impairment.

### Mackworth Clock Test

The Mackworth Clock is an experimental device used in the field of experimental psychology to study the effects of long-term vigilance on the detection of signals. The device has a large black pointer in a large circular background like a clock. The pointer moves in short jumps like the second hand of an analog clock, approximately every second. At infrequent and irregular intervals, the hand makes a double jump, e.g. 12 times every 30 seconds. The task is to detect when the double jumps occur by pressing a button. Typically, Mackworth's participants would do this task for two hours. The Mackworth Clock was used to establish one of the fundamental findings in the vigilance and sustained attention literature: the vigilance decrement (signal detection accuracy) decreases notably after 30 minutes on task. The test continues to be used today in vigilance research in various forms, including computer-displayed versions.

### Montreal Cognitive Assessment (MoCA)

The Montreal Cognitive Assessment (MoCA) is a widely used screening assessment for detecting cognitive impairment. It can help identify those at risk for developing Alzheimer's disease and other forms of dementia. The MoCA contains 30 questions and takes around 10 to 12 minutes to complete. The MoCA Test—was validated as a highly sensitive tool for early detection of mild cognitive impairment (MCI) in 2000. MoCA has been subsequently adopted in clinical settings around the world and is widely used as a scale in academic and non-academic research. The basics of this test include short-term memory, executable performance, attention, focus and more. MoCA scores range between 0 and 30. A score of 26 or over is normal.

### Mini-Mental State Examination (MMSE)

The Mini-Mental State Examination (MMSE) or Folstein test is a 30-point questionnaire that is used extensively in clinical and research settings to measure cognitive impairment. It is commonly used in medicine and allied health to screen for dementia. It is also used to estimate the severity and progression of cognitive impairment and to follow the course of cognitive changes in an individual over time; thus, making it an effective way to document an individual's response to treatment. Administration of the test takes between 5 and 10 minutes and examines functions include simple questions and problems in a number of areas: the time and place of the test, repeating lists of words, arithmetic such as the serial sevens, language use and comprehension, and basic motor skills. Any score of 24 or more (out of 30) indicates normal cognition. Below this, scores can indicate severe (≤9 points), moderate (10-18 points), or mild (19-23 points) cognitive impairment.

The improvements observed after administering Ashwagandha composition are improving memory in adults with mild cognitive impairment (MCI) and without any impairment of Activities of Daily Life, improving subjective complaint of a memory disturbance, reducing memory deficit, improving cognitive functions, improving activities of daily living, reduce dementia. Other improvements are improving the Auditory Immediate index with Verbal long-term memory & verbal learning, Improving the Visual Immediate Index with Nonverbal long-term memory & Nonverbal learning, Improving the Immediate Memory Index, Improving Auditory Delayed Index with Verbal delayed memory & Verbal Learning, Improving Visual Delayed Index with on-verbal delayed memory & Non-verbal Learning, Improving Auditory Recognition Delayed Index with Recognition of verbal information, Improving General Memory, Improving Working Memory with Auditory processing, Attention/concentration Executive function, and Visual-spatial processing.

No significant variability in MOCA is observed with Placebo treatment during day 30 and 60 compared to baseline. Significant improvement is observed with Ashwagandha treatment during day 30 (Percentage change=7.83%) and 60 (Percentage change=14.77%) compared to baseline. Day 60 showed significantly higher MOCA (Percentage change=6.43%) compared to day 30 with Ashwagandha treatment. The result showed an improvement in cognitive impairment after administering with Ashwagandha extract.

No significant variability in MMSE is observed with Placebo treatment during day 30 and 60 compared to baseline. Day 60 showed no significant variability in MMSE compared to day 30 with Placebo treatment. Significant improvement is observed in MMSE with Ashwagandha treatment during day 30 (Percentage change=9.26%) and 60 (Percentage change=19.21%) compared to baseline. The result showed an improvement in cognitive impairment after administering with Ashwagandha extract.

Placebo treated group showed no significant change in Shepard Mental Rotation Task score during day 30 and 60 compared to baseline. Significant improvement is observed with Ashwagandha treatment during day 30 (Percentage change=12.22%) compared to baseline. Day 60 showed significantly higher Shepard mental rotation task (Percentage change=31.67%) compared to baseline with Ashwagandha treatment. Day 60 showed significantly higher (Percentage change=17.37%) Shepard mental rotation task compared to day 30 with Ashwagandha treatment. Results showed an improvement in individual's spatial visualization abilities after administering with Ashwagandha extract.

Significant improvement is observed in Logical memory I (Percentage change—2.63%) and II (Percentage change=4.96%) with Ashwagandha treatment during day 30 compared to baseline. Day 60 showed significantly higher logical memory I (Percentage change=6.5%) and II (Percentage change=12.8%) compared to baseline with Ashwagandha treatment. Day 60 showed significantly higher logical memory I (Percentage change=3.7%) and II (Percentage change=7.5%) compared to day 30 with Ashwagandha treatment. No significant variability was observed with Placebo treatment during day 30 and 60. Results showed an improvement in verbal episodic memory after administering Ashwagandha extract.

Significant improvement in Verbal paired associate I (Percentage change—2.64%) and II (Percentage change=11.02%) is observed with Ashwagandha treatment during day 30 and 60 compared to baseline. Day 60 showed significantly higher VERBAL PAIRED ASSOCIATE I (Percentage change=6.93%) and II (Percentage change=19.21%) compared to day 30 with Ashwagandha treatment. No significant variability is observed with Placebo treatment during day 30 and 60. Results showed an improvement in verbal learning and memory abilities after administering Ashwagandha extract.

No significant variability is observed in FACES I &II with Placebo treatment during day 30 and 60 compared to baseline. A significant variability is observed in FACES I (percentage change=0.73%) & II (percentage change=3.7%) with Ashwagandha treatment during day 30 compared to baseline. Day 60 showed significantly higher FACES I (percentage change=7.06%) and II (percentage change=14.36%) compared to baseline with Ashwagandha treatment. Day 60 showed significantly higher FACES I (percentage change=6.28%) and II (percentage change=10.33%) compared to day 30 with Ashwagandha treatment. Results showed an improvement in visual memory and recognition abilities after administering Ashwagandha extract.

No significant variability in FAMILY PICTURES I and II is observed with Placebo treatment during day 30 and 60 compared to baseline. Significant variability is observed in FAMILY PICTURES I and II with Ashwagandha treatment during day 30 (percentage change=0.5%) and 60 (percentage change=0.25%) compared to baseline. Day 60 showed significantly higher (percentage change=9.25%) FAMILY PICTURES I and FAMILY PICTURES II (percentage change=12.3%) compared to baseline with Ashwagandha treatment. Day 60 showed significantly higher (percentage change=8.706%) FAMILY PICTURES I and II (percentage change=12%) compared to day 30 with Ashwagandha treatment. Results showed an improvement in visual memory and recognition abilities after administering Ashwagandha extract.

No significant variability in Letter number sequencing is observed with Placebo treatment during day 30 (percentage change=0.27%) and day 60 (percentage change=0.53%) compared to baseline. Significant improvement is observed with Ashwagandha treatment during day 30 (percentage change=7.24%) and day 60 (percentage change=16.35%) compared to baseline. Day 60 showed significantly higher LETTER NUMBER SEQUENCING compared to day 30 with Ashwagandha treatment (percentage change=8.5%). Results showed an improvement in working memory and cognitive processing abilities after administering Ashwagandha extract.

No significant variability is observed in SPATIAL SPAN with Placebo treatment during day 30 (percentage change=0.86%) and day 60 (percentage change=1.14%) compared to baseline. Significant improvement is observed with Ashwagandha treatment during day 30 (percentage change=12.9%) and 60 (percentage change=20.8%) compared to baseline. Day 60 showed significantly higher SPATIAL SPAN compared to day 30 with Ashwagandha treatment (percentage change=6.9%). Results showed an improvement in visuospatial working memory and attentional abilities after administering Ashwagandha extract.

No significant improvement is observed in VISUAL REPRODUCTION I and II with Placebo treatment during day 30 and day 60 compared to baseline. Significant improvement is observed in VISUAL REPRODUCTION I (percentage change=7.24%) and II (percentage change—6.1%) with Ashwagandha treatment during day 30 compared to baseline. Day 60 showed significantly higher (percentage change=13.9%) VISUAL REPRODUCTION I and II (percentage change=14.9%) compared to baseline with Ashwagandha treatment. Day 60 showed significantly higher VISUAL REPRODUCTION I (percentage change=6.25%) and II (percentage change=8.25%) compared to day 30 with Ashwagandha treatment. Results showed an improvement in visual memory and visuospatial abilities after administering Ashwagandha extract.

### Example 1

100 kg of roots and leaves of Ashwagandha were collected and cleaned. Cleaned roots and leaves were extracted by refluxing with 80% methanol at the boiling temperature (60-70° C.) of methanol for one hour to obtain the first residue and first supernatant. The first residue was then further extracted two more times with four times the quantity of methanol at each time. The residue and supernatants were separated. All the supernatants were pooled and concentrated in an Agitated thin film evaporator (ATFE) to form a concentrated methanol extract. (Sample 1) (yield 15%).

15 Kg of sample 1 was partitioned with ethyl acetate in liquid-liquid extractor. Aqueous and ethyl acetate layers were formed and each layer was collected separately. The aqueous part was passed through the resin column and elute first with water followed by methanol. Methanol elute was concentrated and dried to form powder of methanol elute of Ashwagandha. (Sample 2, Yield 40%)

Ethyl acetate layer was fractionated by passing through the silica column. Column was eluted with different ratio of hexane:methanol. First fraction (F1) was a elute of 95:5 hexane:methanol (yield 3.8%), second fraction (F2) was a elute of 90:10 hexane:methanol (yield 12.6%), third fraction (F3) was a elute of 85:15 hexane:methanol (yield 15.4%), and final fraction (F4) was a elute of 80:20 hexane:methanol (yield 67.5%), F3 fraction was concentrated and dried to form powder of hexane-methanol elute of Ashwagandha. (sample 3).

Sample 2 and sample 3 were mixed in 9:1 ratio to form Ashwagandha composition (sample 4).

Sample 4 has 2.3% sominone, 23.5% withanolide glycosides, 6.5% Withaferin A and 34.9% withanolides.

### Example 2

Efficacy of Ashwagandha extract on learning and memory (behavioural) activity in experimental rats.

Twenty-four male SD rats were divided into four groups comprising of 6 rats in each group. The animals were treated as per the following details:
Group 1: Normal control (0.5% Polysorbate 80; 10 ml/kg)
Group 2: Untreated control (Scopolamine; 0.6 mg/kg i.p.)
Group 3: Piracetam—200 mg/kg+Scopolamine 0.6 mg/kg i.p.
Group 4: Ashwagandha extract (Sample 4)—100 mg/kg+Scopolamine 0.6 mg/kg i.p.

The vehicle/extract/standard was administered daily for 10 consecutive days. Habituation to swimming was conducted on day 6 of the experiment. Scopolamine was injected 30 minutes after the vehicle/extract/standard to the designated groups. The following behavioural test was conducted at the end of the treatment by using Elevated plus maze apparatus, Y maze model and Cooks pole climbing method.

### Assessment of Memory Activity

The elevated plus-maze consists of two open arms and two closed arms (50×10×40 cm each) were elevated to a height of 50 cm. The rats were placed individually at the end of an open arm facing away from the central platform, and the time taken by them to move from there to either of the close arm transfer latency was recorded. If the animal did not enter into the closed arms within 90 seconds, it was gently pushed into one of the two closed arms and the transferred latency was assigned as 90 seconds. The rats were allowed to explore the maze for another 10 seconds and then return to the home cage. Test substances and Standard drug were administered daily for 10 consecutive days. On the 10th day Standard and test substances were administered orally 30 mins before scopolamine injection transfer latency was recorded.

Pole climbing test: Rats were trained in such a way that the animal had to climb the pole (shock free zone) within 30 s to avoid a shock. The shock was preceded by a buzzer that lasted for 15 s. The animals were trained to climb the pole at the sound of the buzzer (conditioned avoidance response). At particular intervals, 20 trials were given for each animal and average of the shock avoidance and mistakes were recorded. Trained animals were assayed by conditioned avoidance responses.

Y-Maze model—The treatment was started (as per the groups mentioned) for animals from the day 1 and continues up to day 8. On the 8^{th} day, after 30 minutes of amnestic agent induction trails were taken on Y-maze and retention will be observed on 8^{th} day. Briefly, each time animals were placed just inside arm B facing away from the center and allowed to move through apparatus for 8 min, while being monitored by tracking system. Trial was terminated at the specified time duration. Each arm entry (defined as all four paws entering arm) was scored and recorded. Animals were returned to home cage and numbers of fecal pellets were counted in the Y maze and data was recorded. Y-Maze was cleaned with alcohol between trials of each animal.

### Morris Water Maze (MWM) Procedure

The MWM consists of a circular pool (180 cm in diameter and 60 cm in height) with a featureless inner surface. The circular pool was filled with water (23±1° C.) up to a height of 40 cm. The pool was divided into four quadrants of equal area. The escape platform was centered in one of the four quadrants. The day before the training experiments (day 6), animals were habituated to swim training for 60 s in the absence of the platform. The animals were treated with vehicle/extract/standard drug for 6 days. On day 7, 30 min after the administration of vehicle/extract/standard drug, scopolamine (0.6 mg/kg, i.p.) was injected. Training session was started 30 min after scopolamine administration. The rats were given four trial sessions each day for four consecutive days (day 7-10), with an inter-trial interval of 15 min, and the escape latencies were recorded. Once the rat located the platform, it was permitted to remain on it for 15 s to observe the vital clues. If the rat did not locate the platform within 120 s, it was guided to the platform and then allowed to stay on it for 15 s, after that it was removed from the pool. On day 11, the probe test was performed by removing the platform from the pool and a cut-off time of 60 s was kept for exploring. The time spent by the animal in the target quadrant was recorded. The higher value of the time spent in quadrant indicate better retention ability as the animal remembered the position of the platform.

### Results

### Elevated Plus Maze:

The transfer latency of rats of Group 2 got significantly enhanced after the injection of scopolamine as compared to normal control rats. All the extracts as well as the standard drug Piracetam were able to reverse the amnesic effect of scopolamine significantly. The rats treated with Ashwagandha extract (sample 4) have shown transfer latency almost similar to the standard group Piracetam.

TABLE 1
Effect of test products on Transfer latency
of aged rats using elevated plus maze

| Groups | Treatment | Transfer Latency (Sec) |
|---|---|---|
| Group 1 | Normal control | 6.17 ± 1.47*** |
| Group 2 | Untreated control | 35.50 ± 8.29 |
| Group 3 | Reference Standard (Piracetam) | 7.00 ± 0.89*** |
| Group 4 | Ashwagandha extract | 7.50 ± 1.38*** |

Data represented as Mean ± SEM (n = 6). Statistical significance was compared between untreated control (Group 2) versus other groups
(***P Value < 0.0001).

### Pole Climbing Test:

In a pole climbing test, early escape to shock-free zone (avoidance of shock) indicates retention of learned task. As detailed in Table 2, the escape latency of rats of Group 2 got significantly increased (13.83 s) after injection of scopolamine as compared to normal control rats (5.17 s). Prior administration of test product, as well as standard drug Piracetam, were able to reverse the amnesic effect of scopolamine significantly. The rats treated with Ashwagandha extract (sample4) have shown an impressive memory retention effect and it was almost similar to the standard drug Piracetam.

TABLE 2
Effect of test products on avoidance/escape
latency of aged rats using Pole climbing test

| | | Avoidance/Escape |
|---|---|---|
| Groups | Treatment | latency (Sec) |
| Group 1 | Normal control | 5.17 ± 0.75*** |
| Group 2 | Untreated control | 13.83 ± 0.75 |
| Group 3 | Reference Standard (Piracetam) | 6.00 ± 0.89*** |
| Group 4 | Ashwagandha extract | 6.50 ± 1.05*** |

Data represented as Mean ± SEM (n = 6). Statistical significances were compared between untreated control (Group 2) versus other groups
(***P Value < 0.0001).

### Y-Maze Test

The retention of learned task as well as the number of arm entries were observed using Y-Maze in rats. The untreated control rats (Group 2) have shown the highest retention latency and lowest arm entries among all the rats (Table 3). The retention latency was significant in the Ashwagandha extract treated group (2.53 min). The data on the number of arm entries has also shown a significant difference for Ashwagandha extract treated rats (4.50).

TABLE 3
Effect of test products on transfer
latency of aged rats using Y-Maze

| | | Retention Latency | Number of Arm |
|---|---|---|---|
| Groups | Treatment | (Minutes) | entries |
| Group 1 | Normal control | 2.94 ± 0.49*** | 5.17 ± 0.75*** |
| Group 2 | Untreated control | 7.09 ± 0.34 | 1.83 ± 0.75 |
| Group 3 | Reference Standard | 2.07 ± 0.46*** | 5.00 ± 0.89*** |
| | (Piracetam) | | |
| Group 4 | Ashwagandha extract | 2.53 ± 0.32*** | 4.50 ± 0.55*** |

Data represented as Mean ± SEM (n = 6). Statistical significances were compared between untreated control (Group 2) versus other groups
(***P Value < 0.0001).

### Effect on Scopolamine-Induced Amnesia in MWM:

It is evident from the results (Table 4) that normal control group animals showed a significant learning and retention response as indicated by a decrease in the escape latencies (5.67 s) in comparison to that of scopolamine-treated animals (57.92 s) on day 10. Pre-treatment with Ashwagandha extract at 100 mg/kg in rats exhibited amelioration of scopolamine-induced amnesia. The ameliorating effect of Ashwagandha extract was evident from the significant (p<0.05) decrease in the escape latency (7.17 s) as compared to scopolamine-treated group (57.92 s) on day 10. Piracetam also significantly (p<0.05) reversed the scopolamine induced amnesia as evident from the decreased escape latency (5.25 s) as compared to the scopolamine-treated group.

TABLE 4
Time to reach escape platform on training
days (Mean value in seconds)

| Groups | Treatment | Day 7 | Day 8 | Day 9 | Day 10 |
|---|---|---|---|---|---|
| Group 1 | Normal control | 44.50 | 17.17 | 7.58 | 5.67 |
| Group 2 | Untreated control | 120.00 | 110.50 | 93.17 | 57.92 |
| Group 3 | Reference Standard | 74.25 | 47.33 | 9.83 | 5.25 |
| | (Piracetam) | | | | |
| Group 4 | Ashwagandha extract | 76.08 | 24.67 | 11.42 | 7.17 |

From the above results, it is evident that the Ashwagandha-treated group exhibited better activities on the enhancement of learning and memory (behavioural) than other groups when compared to the untreated control group.

### Example 3

40 healthy subjects with mild cognitive impairment were randomized in a 1:1 (n=20 each) ratio to receive treatment with either ashwagandha extract with 2% sominone (250 mg once daily) or a placebo for consecutive 60 days.
Group 1: Ashwagandha extract (sample 4) (Dosage 250 mg once daily for 60 days)
Group 2: Placebo (Dosage 250 mg once daily for 60 days)

The primary outcomes were the change (baseline to 30-Day and 60-Day) in cognition function assessed using outcome measures, Wechsler Memory Scale-III (WMS-III), and Shepard Mental Rotation Task.

The secondary outcomes were the change (baseline to 30-Day and 60-Day) in MoCA and MMSE scores.

Within group analysis (Placebo Group and Ashwagandha group)—Comparison Between Day 30-Baseline, Day 60 to Baseline and Day 60 to Day 30.

### Montreal Cognitive Assessment (MOCA)

No significant variability was observed with Placebo treatment during day 30 compared to baseline (percentage change=−0.45%). Day 60 showed no significant variability in MOCA compared to baseline with Placebo treatment (percentage change=0.90%). Day 60 showed no significant variability in MOCA task compared to day 30 with Placebo treatment (percentage change=1.36%). Significant improvement was observed with Ashwagandha treatment during day 30 compared to baseline (percentage change=7.83%). Day 60 showed significantly higher MOCA compared to baseline with Ashwagandha treatment (percentage change=14.77%). Day 60 showed significantly higher MOCA compared to day 30 with Ashwagandha treatment (percentage change=6.43%). The result showed an improvement in cognitive impairment after administering with Ashwagandha extract. Above results are represented as FIG. 1.

### Mini-Mental State Examination (MMSE)

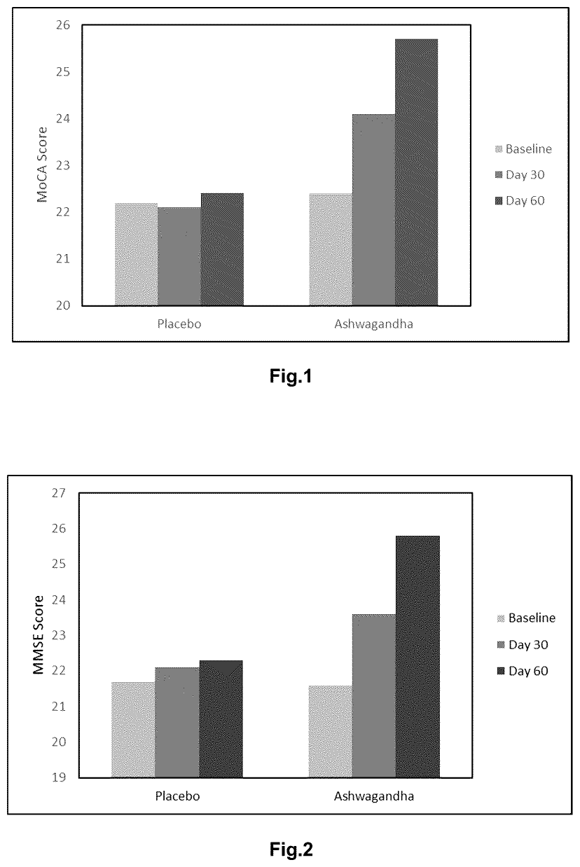

No significant variability was observed with Placebo treatment during day 30 and 60 compared to baseline (percentage change=2.08% and 2.77% respectively). Day 60 showed no significant variability in MMSE compared to day 30 with Placebo treatment (percentage change=0.68%). Significant improvement was observed with Ashwagandha treatment during day 30 compared to baseline (percentage change=9.26%). Day 60 showed significantly higher MMSE compared to baseline with Ashwagandha treatment (percentage change=19.21%). Day 60 showed significantly higher MMSE compared to day 30 with Ashwagandha treatment (percentage change=9.11%). The result showed an improvement in cognitive impairment after administering with Ashwagandha extract. Above results are represented as FIG. 2.

### Shepard Mental Rotation Task

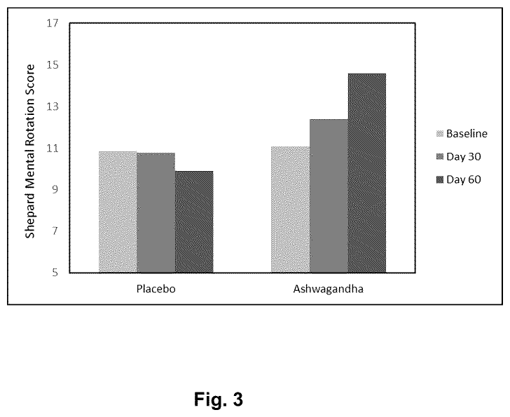

No significant variability was observed with Placebo treatment during day 30 compared to baseline (percentage change=−0.46%). Day 60 showed significantly lower Shepard mental rotation task compared to baseline with Placebo treatment (percentage change=−9.216%). Day 60 showed significantly lower Shepard mental rotation task compared to day 30 with Placebo treatment (percentage change=−8.796%). Significant improvement was observed with Ashwagandha treatment during day 30 compared to baseline (percentage change=12.22%). Day 60 showed significantly higher Shepard mental rotation task compared to baseline with Ashwagandha treatment (percentage change=31.67%). Day 60 showed significantly higher Shepard mental rotation task compared to day 30 with Ashwagandha treatment (percentage change=17.34%). Results showed an improvement in individual's spatial visualization abilities after administering with Ashwagandha extract. Above results are represented as FIG. 3.

### Wechsler Memory Scale-III (WMS III)—Immediate Memory

a. Logical Memory I

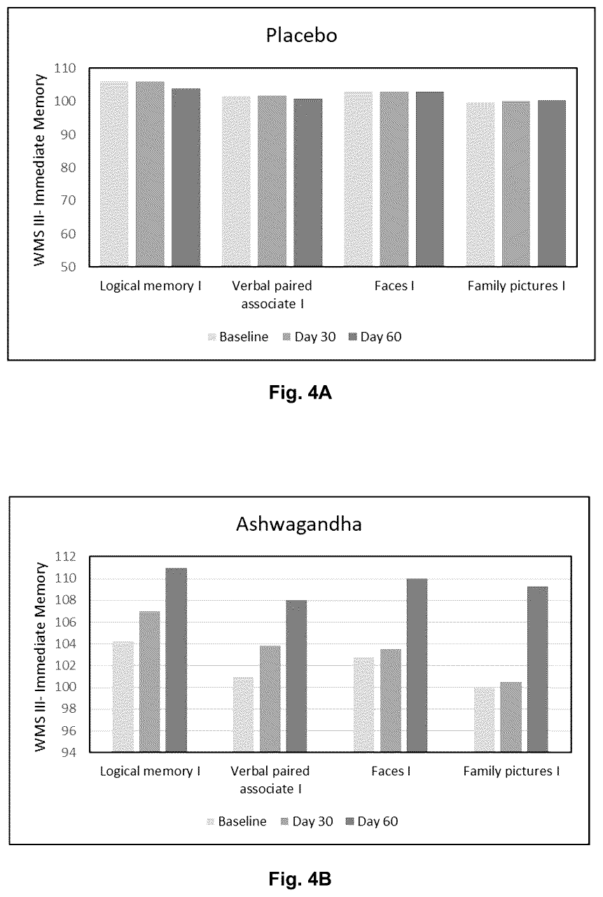

Significant improvement was observed with Ashwagandha treatment during day 30 compared to baseline. There was a 2.63% improvement in logical memory at day 30 compared to baseline. Day 60 showed significantly higher (6.5%) logical memory I compared to baseline with Ashwagandha treatment. Day 60 showed significantly higher logical memory I (3.7%) compared to day 30 with Ashwagandha treatment. No significant variability was observed with Placebo treatment during day 30 (−0.235%) and day 60 (−2.117%) compared to baseline. Day 60 showed significant reduction in LOGICAL MEMORY I compared to day 30 with Placebo treatment (percentage change=−1.886%). Results showed an improvement in verbal episodic memory after administering Ashwagandha extract. Above results are represented as FIGS. 4A and 4B.

b. Verbal Paired Associate I

Significant improvement (2.64%) was observed with Ashwagandha treatment during day 30 compared to baseline. Day 60 showed significantly higher VERBAL PAIRED ASSOCIATE I compared to baseline with Ashwagandha treatment (percentage change 6.93%). Day 60 showed significantly higher VERBAL PAIRED ASSOCIATE I compared to day 30 with Ashwagandha treatment (percentage change 4.1%).

No significant variability was observed with Placebo treatment during day 30 (0% change) compared to baseline. Day 60 showed no significant variability in VERBAL PAIRED ASSOCIATE I compared to baseline with Placebo treatment (percentage change=−0.982%). Day 60 showed significant reduction in VERBAL PAIRED ASSOCIATE I compared to day 30 with Placebo treatment (percentage change=−0.982%). Results showed an improvement in verbal learning and memory abilities after administering Ashwagandha extract. Above results are represented as FIGS. 4A and 4B.

c. Faces I

No significant variability was observed with Placebo treatment during day 30 (−0.242%) and 60 (−0.242%) compared to baseline. Day 60 showed no significant variability in FACES I compared to day 30 with Placebo treatment (percentage change=0%). No significant variability was observed with Ashwagandha treatment during day 30 compared to baseline (percentage change=0.729%). Day 60 showed significantly higher FACES I compared to baseline with Ashwagandha treatment (percentage change=7.06%). Day 60 showed significantly higher FACES I compared to day 30 with Ashwagandha treatment (percentage change 6.280%). Results showed an improvement in visual memory and recognition abilities after administering Ashwagandha extract. Above results are represented as FIGS. 4A and 4B.

d. Family Pictures I

No significant variability in FAMILY PICTURES I was observed with Placebo treatment during day 30 (percentage change −0.25%) and 60 (percentage change=0.5%) compared to baseline. Day 60 showed no significant variability in FAMILY PICTURES I compared to day 30 with Placebo treatment (percentage change=0.249%). Significant variability was observed with Ashwagandha treatment during day 30 compared to baseline (percentage change=0.5%). Day 60 showed significantly higher (percentage change=9.25%) FAMILY PICTURES I compared to baseline with Ashwagandha treatment. Day 60 showed significantly higher (percentage change=8.706%) FAMILY PICTURES I compared to day 30 with Ashwagandha treatment. Results showed an improvement in visual memory and recognition abilities after administering Ashwagandha extract. Above results are represented as FIGS. 4A and 4B.

### Wechsler Memory Scale-III (WMS III)—General Memory

a. Logical Memory II

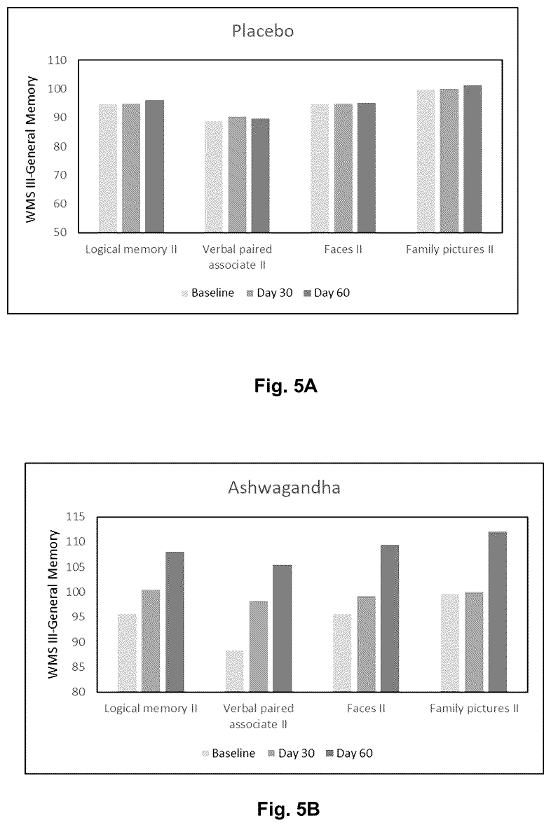

Significant improvement (percentage change=4.96%) was observed in LOGICAL MEMORY II with Ashwagandha treatment during day 30 compared to baseline. Day 60 showed significantly higher (12.8%) logical memory II compared to baseline with Ashwagandha treatment. Day 60 showed significantly higher logical memory II (7.5%) compared to day 30 with Ashwagandha treatment. No significant variability was observed in LOGICAL MEMORY II with Placebo treatment during day 30 (0.263% %) and day 60 (1.319%) compared to baseline. Day 60 showed significant reduction in LOGICAL MEMORY II compared to day 30 with Placebo treatment (percentage change=1.052%). Results showed an improvement in verbal episodic memory after administering Ashwagandha extract. Above results are represented as FIGS. 5A and 5B.

b. Verbal Paired Associate II

Significant improvement (11.02%) in Verbal paired associate II was observed with Ashwagandha treatment during day 30 compared to baseline. Day 60 showed significantly higher VERBAL PAIRED ASSOCIATE II compared to baseline with Ashwagandha treatment (percentage change=19.21%). Day 60 showed significantly higher VERBAL PAIRED ASSOCIATE II compared to day 30 with Ashwagandha treatment (percentage change=7.38%).

No significant variability was observed with Placebo treatment during day 30 (1.40% change) compared to baseline. Day 60 showed no significant variability in VERBAL PAIRED ASSOCIATE II compared to baseline with Placebo treatment (percentage change=2.53%). Day 60 showed significant reduction in VERBAL PAIRED ASSOCIATE II compared to day 30 with Placebo treatment (percentage change=1.11%). Results showed an improvement in verbal learning and memory abilities after administering Ashwagandha extract. Above results are represented as FIGS. 5A and 5B.

c. Faces II

No significant variability was observed with Placebo treatment during day 30 (1.32%) compared to baseline. Day 60 showed no significant improvement in FACES II compared to baseline with Placebo treatment. Day 60 showed no significant variability in FACES II compared to day 30 with Placebo treatment (percentage change=1.8%). Significant improvement was observed with Ashwagandha treatment during day 30 compared to baseline (percentage change=3.7%). Day 60 showed significantly higher FACES II compared to baseline with Ashwagandha treatment (percentage change=14.36%). Day 60 showed significantly higher FACES II compared to day 30 with Ashwagandha treatment (percentage change=10.33%). Results showed an improvement in visual memory and recognition abilities after administering Ashwagandha extract. Above results are represented as FIGS. 5A and 5B.

d. Family Pictures II

No significant variability in FAMILY PICTURES II was observed with Placebo treatment during day 30 (percentage change 0%) and 60 (percentage change=1.3%) compared to baseline. Day 60 showed no significant variability in FAMILY PICTURES II compared to day 30 with Placebo treatment (percentage change=1.25%). Significant variability was observed with Ashwagandha treatment during day 30 compared to baseline (percentage change=0.25%). Day 60 showed significantly higher (percentage change=12.3%) FAMILY PICTURES II compared to baseline with Ashwagandha treatment. Day 60 showed significantly higher (percentage change=12%) FAMILY PICTURES II compared to day 30 with Ashwagandha treatment. Results showed an improvement in visual memory and recognition abilities after administering Ashwagandha extract. Above results are represented as FIGS. 5A and 5B.

### Wechsler Memory Scale-III (WMS III)—Working Memory

a. Letter Number Sequencing

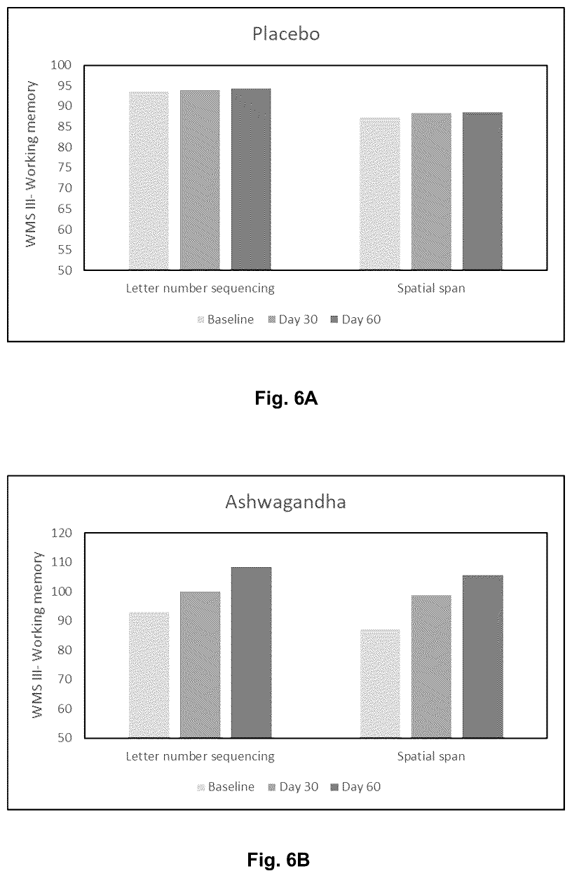

No significant variability in Letter number sequencing was observed with Placebo treatment during day 30 (percentage change=0.27%) and day 60 (percentage change=0.53%) compared to baseline. Day 60 showed no significant variability in LETTER NUMBER SEQUENCING compared to day 30 with Placebo treatment (percentage change=0.27%). Significant improvement was observed with Ashwagandha treatment during day 30 (percentage change=7.24%) and day 60 (percentage change=16.35% compared to baseline. Day 60 showed significantly higher LETTER NUMBER SEQUENCING compared to day 30 with Ashwagandha treatment (percentage change=8.5%). Results showed an improvement in working memory and cognitive processing abilities after administering Ashwagandha extract. Above results are represented as FIGS. 6A and 6B.

b. Spatial Span

No significant variability was observed in SPATIAL SPAN with Placebo treatment during day 30 (percentage change=0.86%) and day 60 (percentage change=1.14%) compared to baseline. Day 60 showed no significant variability in SPATIAL SPAN compared to day 30 with Placebo treatment (percentage change=0.28%). Significant improvement was observed with Ashwagandha treatment during day 30 compared to baseline (percentage change=12.9%). Day 60 showed significantly higher SPATIAL SPAN compared to baseline with Ashwagandha treatment (percentage change=20.6%). Day 60 showed significantly higher SPATIAL SPAN compared to day 30 with Ashwagandha treatment (percentage change=6.84%). Results showed an improvement in visuospatial working memory and attentional abilities after administering Ashwagandha extract. Above results are represented as FIGS. 6A and 6B.

### Wechsler Memory Scale-III (WMS III)—Visuospatial Processing and Response

a. Visual Reproduction I

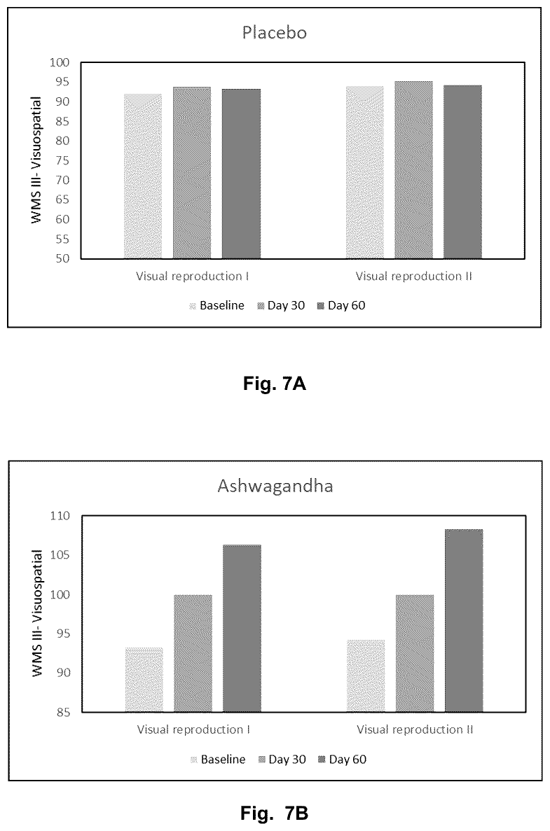

No significant improvement was observed in VISUAL REPRODUCTION with Placebo treatment during day 30 (percentage change=1.626%) and day 60 (percentage change=1.084%) compared to baseline. Day 60 showed no significant variability in VISUAL REPRODUCTION compared to day 30 with Placebo treatment (percentage change=−0.533%). Significant improvement was observed with Ashwagandha treatment during day 30 compared to baseline (percentage change=7.24%). Day 60 showed significantly higher (percentage change=13.941%) VISUAL REPRODUCTION compared to baseline with Ashwagandha treatment. Day 60 showed significantly higher VISUAL REPRODUCTION compared to day 30 with Ashwagandha treatment (percentage change=6.25%). Above results are represented as FIGS. 7A and 7B.

b. Visual Reproduction II

No significant variability was observed in VISUAL REPRODUCTION II with Placebo treatment during day 30 (percentage change=1.061%) and day 60 (percentage change=0%) compared to baseline. Day 60 showed no significant variability in VISUAL REPRODUCTION II compared to day 30 with Placebo treatment (percentage change=−104%). Significant improvement was observed with Ashwagandha treatment during day 30 compared to baseline (percentage change=6.1%). Day 60 showed significantly higher VISUAL REPRODUCTION II compared to baseline with Ashwagandha treatment (percentage change=14.854%). Day 60 showed significantly higher VISUAL REPRODUCTION II compared to day 30 with Ashwagandha treatment (percentage change=8.25%). Above results are represented as FIGS. 7A and 7B.

Results showed an improvement in visual memory and visuospatial abilities after administering Ashwagandha extract.

### Between Group Analysis for Ashwagandha Group and Placebo.

### Wechsler Memory Scale-III (WMS III)

a. Logical Memory I

Ashwagandha treatment showed significantly higher Logical memory I than Placebo treatment during the observations at day 30 (mean difference (Ashwagandha−Placebo)=3). During day 60, Ashwagandha treatment showed significantly higher Logical memory I than Placebo treatment (mean difference (Ashwagandha−Placebo)=9).

b. Verbal Paired Associate I

Ashwagandha treatment showed significantly higher Verbal paired associate I than Placebo treatment during the observations at day 30 (mean difference (Ashwagandha−Placebo)=2.75). During day 60, Ashwagandha treatment showed significantly higher Verbal paired associate I than Placebo treatment (mean difference (Ashwagandha−Placebo)=8).

c. Faces I

Ashwagandha treatment showed significantly higher Faces I than Placebo treatment during the observations at day 30 (mean difference (Ashwagandha−Placebo)=1). During day 60, Ashwagandha treatment showed significantly higher Faces I than Placebo treatment (mean difference (Ashwagandha−Placebo)=7.5).

d. Family Pictures I

Ashwagandha treatment showed no significant variability in Family pictures I compared to Placebo treatment during the observations at day 30 (mean difference (Ashwagandha−Placebo)=0.25). During day 60, Ashwagandha treatment showed significantly higher Family pictures I than Placebo treatment (mean difference (Ashwagandha−Placebo)=8.75).

e. Logical Memory II

Ashwagandha treatment showed significantly higher Logical memory II than Placebo treatment during the observations at day 30 (mean difference (Ashwagandha−Placebo)=4.5). During day 60, Ashwagandha treatment showed significantly higher Logical memory II than Placebo treatment (mean difference (Ashwagandha−Placebo)=11).

f. Verbal Paired Associate II

Ashwagandha treatment showed significantly higher Verbal paired associate II than Placebo treatment during the observations at day 30 (mean difference (Ashwagandha−Placebo)=6.083). During day 60, Ashwagandha treatment showed significantly higher Verbal paired associate II than Placebo treatment (mean difference (Ashwagandha−Placebo)=14.75).

g. Faces II

Ashwagandha treatment showed no significant variability in Faces II compared to Placebo treatment during the observations at day 30 (mean difference (Ashwagandha−Placebo)=2.25). During day 60, Ashwagandha treatment showed significantly higher Faces II than Placebo treatment (mean difference (Ashwagandha−Placebo)=10.75).

h. Family Pictures II

Ashwagandha treatment showed no significant variability in Family pictures II compared to Placebo treatment during the observations at day 30 (mean difference (Ashwagandha−Placebo)=0.25). During day 60, Ashwagandha treatment showed significantly higher Family pictures II than Placebo treatment (mean difference (Ashwagandha−Placebo)=11).

### Letter Number Sequencing

Ashwagandha treatment showed significantly higher Letter number sequencing than Placebo treatment during the observations at day 30 (mean difference (Ashwagandha−Placebo)=6.5). During day 60, Ashwagandha treatment showed significantly higher Letter number sequencing than Placebo treatment (mean difference (Ashwagandha−Placebo)=14.75).

### Spatial Span

Ashwagandha treatment showed significantly higher Spatial span than Placebo treatment during the observations at day 30 (mean difference (Ashwagandha−Placebo)=6.5). During day 60, Ashwagandha treatment showed significantly higher Spatial span than Placebo treatment (mean difference (Ashwagandha−Placebo)=14.75).

### Visual Reproduction I

Ashwagandha treatment showed significantly higher Visual reproduction I than Placebo treatment during the observations at day 30 (mean difference (Ashwagandha−Placebo)=5.25). During day 60, Ashwagandha treatment showed significantly higher Visual reproduction I than Placebo treatment (mean difference (Ashwagandha−Placebo)=12).

### Visual Reproduction II

Ashwagandha treatment showed significantly higher Visual reproduction II than Placebo treatment during the observations at day 30 (mean difference (Ashwagandha−Placebo)=4.75). During day 60, Ashwagandha treatment showed significantly higher Visual reproduction II than Placebo treatment (mean difference (Ashwagandha−Placebo)=14).

### Shepard Mental Rotation

Ashwagandha (Ashwagandha) treatment showed significantly higher Shepard mental rotation than Placebo treatment during the observations at day 30 (mean difference (Ashwagandha-Placebo)=1.4). During day 60, Ashwagandha treatment showed significantly higher Shepard mental rotation than Placebo treatment (mean difference (Ashwagandha−Placebo)=4.5).

### MoCA

Ashwagandha treatment showed significantly higher MoCA than Placebo treatment during the observations at day 30 (mean difference (Ashwagandha−Placebo)=1.8). During day 60, Ashwagandha treatment showed significantly higher MoCA than Placebo treatment (mean difference (Ashwagandha−Placebo)=3.1).

### MMSE

Ashwagandha treatment showed significantly higher MMSE than Placebo. treatment during the observations at day 30 (mean difference (Ashwagandha−Placebo)=1.8). During day 60, Ashwagandha treatment showed significantly higher MMSE than Placebo treatment (mean difference (Ashwagandha−Placebo)=3.1).

From the above result, increased score of WMS-III subtests indicates a significant improvement in immediate, long-term and working memory with Ashwagandha treatment as compared to placebo. Visuospatial performance was also increased after treating with Ashwagandha extract. Ashwagandha treatment showed increase in MMSE score supporting improvement in cognitive functions. In the above study, the MoCA score was significantly enhanced at 30 and 60-Day with Ashwagandha treatment as compared to placebo.

The reduced visuospatial working memory is an early sign of MCI which may be due to shrinkage/atrophy of the hippocampus led to deleterious effect on visual-grapheme-phoneme conversion. The significant improvement in visuospatial working memory in the above study indicate positive effect of Ashwagandha on visual-grapheme-phoneme conversion at hippocampus fusiform gyrus. Studies have reported that improved working memory results in the effective conversion of short-term memory into long-term memory (Alescio-Lautier, 2007; Missonnier, 2007; Yetkin, 2006).

The above results suggest that the therapy with Ashwagandha standardised with sominone has led to significant improvements in enhancing both immediate and general memory in people with Mild Cognitive Impairment (MCI) as well as improving cognitive function, such as attention, and information processing speed.

1-27. (canceled)

28. A medicinal composition derived from Withania somnifera extract comprising withanolides and sominone wherein said sominone in the Withania somnifera extract is standardized in the range of 0.1-30%.

29. The medicinal composition of claim 28, wherein withanolides are in the range of 10-60%.

30. The medicinal composition of claim 28 further comprising withanolide glycosides and withaferin A.

31. The medicinal composition of claim 30, wherein withanolide glycosides is in the range of 10-25%, and withaferin A is in the range of 2-10%.

32. The medicinal composition of claim 30, wherein said composition comprises at least 2% sominone, at least 20% withanolide glycosides, at least 6% withaferin A and at least 30% withanolides.

33. The medicinal composition of claim 28 further comprising a delivery system, wherein the delivery system comprises a pharmaceutically acceptable excipient selected from the group consisting of a diluent, a binder, a wetting agent, a disintegrant, a lubricant and a mixture thereof.

34. A method of preparing a medicinal composition derived from a Withania somnifera extract comprising the steps of
a) cleaning an Ashwagandha plant part (root and leaf);
(b) drying the cleaned plant part and powdering it;
(c) extracting the powdered part using a polar solvent;
(d) collecting the supernatant after extracting and followed by concentrating to obtain a concentrate;
(e) subjecting said concentrate to liquid-liquid extraction using a nonpolar solvent;
(f) separating an aqueous and a solvent part after the liquid-liquid extraction;
(g) subjecting the aqueous part in step (f) to column purification using water and methanol;
(h) collecting the water elute and the methanol elute and keeping aside;
(i) subjecting the solvent part collected in step (f) to column purification by eluting with hexane-methanol in different ratios and collecting different fractions;
(j) blending the methanol elute of step (h) and one of the fractions from step (i) containing a higher percentage of actives to obtain the extract of Withania somnifera, and
(k) preparing the medicinal composition comprising the extract of Withania somnifera.

35. The method of preparing the medicinal composition of claim 34, wherein a blending ratio of the methanol elute of step (h) and one of the fractions from step (i) containing the higher percentage of actives ranges from 20:1 to 2:1 and preferably 9:1.

36. A dosage form comprising a medicinal composition derived from a Withania somnifera extract, wherein the Withania somnifera extract comprises withanolides and sominone, and wherein the sominone in the extract is standardized in the range of 0.1% to 30%.

37. The dosage form of claim 36, wherein said medicinal composition further comprises withanolide glycosides and withaferin A.

38. The dosage form of claim 37, wherein withanolide glycosides is in the range of 10-25% and withaferin A is in the range of 2-10%.

39. The dosage form of claim 38 comprising 2.3% sominone, 23.5% withanolide glycosides and 6.5% withaferin A, and 34.9% withanolides.

40. The dosage form of claim 37, further comprising a delivery system, wherein the delivery system comprises a pharmaceutically acceptable excipient selected from the group consisting of a diluent, a binder, a wetting agent, a disintegrant, a lubricant and a mixture thereof.

41. The dosage form of claim 36, wherein a dose is administered to a subject, and wherein the dose ranges from about 30 mg to about 2000 mg per day.

42. The dosage form of claim 36, selected from a group consisting of a capsule, a tablet, a mini tablet, a granule, a sachet, a powder, a paste, an infusion, an injection, an ampoule, a solution, a suspension, an emulsion, a pill, and a cream.

43. The dosage form of claim 36, wherein the dosage form enhances the bioavailability of its constituents when administered to a subject in need thereof.

44. A method of improving a condition in a subject in need thereof, the method comprising administering the dosage form of claim 36, wherein the condition is selected from the group consisting of learning and behavioral activity; immediate memory, general memory, working memory and visuospatial memory; verbal episodic memory; verbal learning and memory abilities; visual memory and recognition abilities; working memory and cognitive processing abilities; visuospatial working memory and attentional abilities by improvement in Spatial span score; visual memory and visuospatial abilities; individual's spatial visualization abilities assessed by increase in Shepard Mental Rotation Task score; and cognitive impairment by improving in Montreal cognitive assessment (MoCA) score and Mini-Mental State Examination (MMSE) scores, and combinations thereof.
